(19) United States
(12) Patent Application Publication
(10) Pub. No.: US 2022/0339573 A1
(43) Pub. Date: Oct. 27, 2022

(54) AIR CLEANER

(71) Applicant: MIRO CO., LTD., Incheon (KR)

(72) Inventors: Dong Jin Seo, Incheon (KR); Yong Ju Oh, Incheon (KR); Min Seok Kim, Incheon (KR); Jeong Won Lee, Incheon (KR)

(73) Assignee: MIRO CO., LTD., Incheon (KR)

(21) Appl. No.: 17/763,853

(22) PCT Filed: Feb. 9, 2021
(86) PCT No.: PCT/KR2021/001750

§ 371 Date: Mar. 25, 2022

(30) Foreign Application Priority Data
Aug. 13, 2020 (KR) 10-2020-0101514
Oct. 22, 2020 (KR) 10-2020-0137414

(51) Int. Cl. B01D 46/00; B01D 46/24; F24F 8/10; F24F 8/22; A61L 9/20

(52) U.S. Cl. CPC B01D 46/0028; B01D 46/0049; B01D 46/0043; B01D 46/0002; B01D 46/2403; F24F 8/10; F24F 8/22; A61L 9/20; B01D 2273/30; B01D 2279/65; A61L 2209/14; A61L 2209/12

(57) ABSTRACT

Various embodiments of the present invention disclosed herein relate to an air cleaner, and may suck outside air and purify and sterilize the outside air, and may perform sterilization of a filter. An embodiment of the invention disclosed herein discloses an air cleaner including a body forming an appearance; a suction unit formed on the body and introduced with outside air; a discharge unit formed on the body and discharging the air introduced through the suction unit; a fan module provided inside the body and forming a flow of air introduced into the suction unit and discharged to the discharge unit; a filter unit provided inside the body to filter the air introduced into the suction unit; and a light source unit provided inside the body and irradiating light toward an inner side surface of the filter unit.

### TECHNICAL FIELD

The present invention relates to an air cleaner having an air purifying function and an air sterilizing function.

### BACKGROUND ART

An air cleaner is understood as a device that sucks in outside air, filters out pollutants contained in the outside air, and then discharges the pollutants. The air cleaner may include a blower for introducing external air into the air cleaner and a filter capable of filtering out dust, bacteria, or the like contained in the external air.

In general, the air cleaner is provided with a suction port and a discharge port for introducing and discharging air. In addition, a blower for air circulation, a filter for filtering out pollutants contained in the introduced air, etc., may be provided inside the air cleaner.

Reviewing an operating principle of the air cleaner including the above-described configuration, the air cleaner sucks outside air into the suction port through airflow generated while the blower is driven, filters out pollutants contained in the sucked air through filters of various materials provided inside the air cleaner, and discharges air from which the pollutants are filtered to the outside through the discharge port.

The air purifier is understood as a device that sucks the outside air, sterilizes bacteria contained in the outside air, and then discharges the sterilized bacteria.

In general, the air purifier may be distinguished from an air cleaner that filters out pollutants contained in the outside air through the filter in that it sterilizes the bacteria contained in the outside air. The air purifier generally sterilizes air sucked from the outside through ozone, ions, ultraviolet rays, photocatalysts, and the like.

Recently, in order to supply clean air, an air cleaner capable of performing the functions of the air purifier such as purifying air through a filter method using various filters or a plasma method using ions has been proposed.

However, in order to increase the sterilization effect of air, since the sterilization of the air should be continued for an effective time, the flow of air should be made slowly, but the faster the air flow, the lower the sterilization effect. On the other hand, in order to effectively filter out foreign substances contained in the air, because it has to go through circulation processes several times, the faster the air flow, the greater the filtering effect, and the slower the air flow, the lower the filtering effect.

Therefore, in the case of an air cleaner that has been proposed recently, it is possible to remove some of the pollutants or harmful substances contained in the air and sterilize the bacteria contained in the air to a certain extent, but there is a limit in completely sterilize the bacteria contained in the air.

In addition, in order to improve the sterilization ability of air, a device for sterilizing air in which a photocatalyst material and a UV generator are combined has been developed. Examples of the device include Prior Document No. 10-0660138 (registered on Dec. 14, 2006), and Related Document No. 10-0943328 (registered on Feb. 11, 2010).

However, the air sterilizers described in the above-described prior documents and presented recently are difficult to replace, repair, and clean parts, and therefore, inconvenient to perform follow-up management because the sterilization device that sterilizes and deodorizes introduced air and discharges the introduced air is formed integrally with a main body. In addition, in the structure of the related art document, the air purifying performance is inferior and the sterilization of the filter for air cleaning may not be performed, so there is a limit in enhancing the air sterilization effect.

### DISCLOSURE

### Technical Problem

Accordingly, an object of the present invention is to solve the above-described problem.

Another object of the present invention provides an air cleaner capable of sterilizing a filter while improving sterilization ability of sucked air.

Another object of the present invention provides an air cleaner of various structures provided inside an air cleaner to improve sterilization power of sucked air.

Another object of the present invention provides an air cleaner having a structure in which a user can easily maintain a sterilization module provided inside an air cleaner.

Another object of the present invention provides an air cleaner having a structure that can maximize a sterilization area of a sterilization module provided inside an air cleaner.

### Technical Solution

In an aspect of the present invention, an air cleaner includes a body forming an appearance; a suction unit formed on the body and introduced with outside air; a discharge unit formed on the body and discharging the air introduced through the suction unit; a fan module provided inside the body and forming a flow of air introduced into the suction unit and discharged to the discharge unit; a filter unit provided inside the body to filter the air introduced into the suction unit; and a light source unit provided inside the body and irradiating light toward an inner side surface of the filter unit.

The body may include a first body having a discharge unit formed on one surface and forming a space for accommodating the fan module, and a second body having a suction unit formed on one surface and forming a space for accommodating the filter unit and the light source unit.

The first body may be separably coupled to an upper portion of the second body.

The suction unit may be formed along an outer circumference of the second body, and the filter unit may be spaced apart from the suction unit by a predetermined distance inside the second body and may be provided at a position facing the suction unit.

The air cleaner may further include a base separably coupled to a lower portion of the second body to support the air cleaner.

The light source unit may be coupled to the first body to extend by a predetermined length toward the base.

The light source units may be coupled to an inner side of the second body and may each be provided at positions facing each other with respect to a central axis of the base.

The light source unit may be provided on the base.

The base may include a protrusion formed to protrude toward the first body, and the light source unit may be provided in the protrusion.

The protrusion may include a recession that is recessed from an upper surface of the protrusion toward the base, and the light source unit may be provided on a side surface forming the recession.

The second body may be provided in a cylindrical shape, the filter unit may be provided in a cylindrical shape corresponding to a curvature of the second body, and the light source unit may be positioned in a hollow formed by the second body.

The light source unit may be coupled to the first body and may be provided in a cylindrical shape extending by a predetermined length.

Central axes of the second body, the filter unit, and the light source unit may be the same.

Each of the features of the above-described embodiments may be implemented in combination in other embodiments as long as they are not inconsistent with or exclusive to other embodiments.

### Advantageous Effects

According to various embodiments of the present invention, it is possible to simultaneously clean and sterilize air in a targeted space, and irradiate light generated from a sterilization module over a front surface of a filter directly to prevent air cleaning ability from being lowered when sterilizing the air.

According to various embodiments of the present invention, it is possible to sterilize a filter while improving sterilization ability of sucked air.

According to various embodiments of the present invention, it is possible to facilitate maintenance of a sterilization module provided inside an air cleaner and easily replace a filter.

According to various embodiments of the present invention, it is possible to maximize a sterilization area of a sterilization module provided inside an air cleaner.

The effects of the present invention are not limited to the above-mentioned effects, and other effects that are not mentioned may be obviously understood by those skilled in the art from the following description.

### BRIEF DESCRIPTION OF THE DRAWINGS

FIG. 1 is a perspective view illustrating an air cleaner according to an embodiment of the present invention.

FIG. 2 is a diagram illustrating a side surface of FIG. 1.

FIG. 3 is a diagram illustrating a side cross-sectional surface of FIG. 1.

FIG. 4 is a diagram illustrating a first body of FIG. 1.

FIG. 5 is a diagram illustrating a second body of FIG. 1.

FIG. 6 is a diagram illustrating an embodiment of coupling and detachment of the first body and the second body of FIG. 1.

FIG. 7 is a side cross-sectional view of an air cleaner to which a light source unit according to another embodiment of the present invention is applied.

FIG. 8 is a side cross-sectional view of an air cleaner to which a light source unit according to another embodiment of the present invention is applied.

FIG. 9 is a side cross-sectional view of an air cleaner to which a light source unit according to another embodiment of the present invention is applied.

FIG. 10 is a diagram illustrating a base of FIG. 9.

FIG. 11 is a diagram illustrating a sterilization area of a filter by the light source unit of FIG. 9.

### BEST MODE

Hereinafter, exemplary embodiments of the present invention will be described with reference to the accompanying drawings. The following detailed description is provided to help a comprehensive understanding of methods, apparatuses, and/or systems described herein. However, this is by way of examples only and the present invention is not limited thereto.

In describing the embodiments of the present invention, when a detailed description of well-known technology relating to the present invention may unnecessarily make unclear the spirit of the present invention, a detailed description thereof will be omitted. Further, the following terminologies are defined in consideration of the functions in the present invention and may be construed in different ways by the intention of users and operators. Therefore, the definitions thereof should be construed based on the contents throughout the specification. The terminologies used in the detailed description are for the purpose of describing embodiments of the present invention only, and should in no way limit the present invention. Unless explicitly used otherwise, expressions in the singular include the meaning of the plural. In this description, expressions such as “including” or “comprising” are intended to indicate certain characteristics, numbers, steps, acts, elements, some or a combination thereof, and should not be construed to exclude the presence or possibility of one or more other features, numbers, steps, operations, elements, or some or any combination thereof other than those described.

In addition, terms ‘first’, ‘second’, A, B, (a), (b), and the like, will be used in describing components of exemplary embodiments of the present disclosure. These terms are used only in order to distinguish any component from other components, and features, sequences, or the like, of corresponding components are not limited by these terms.

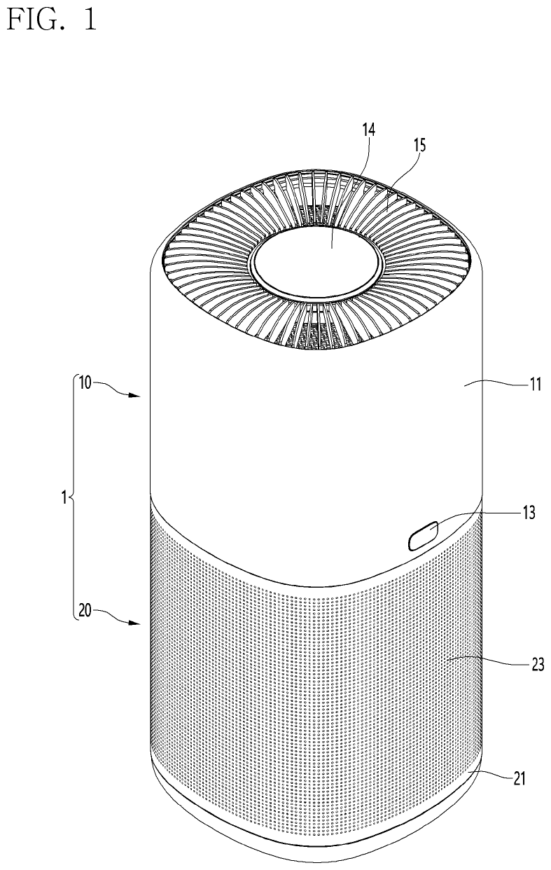

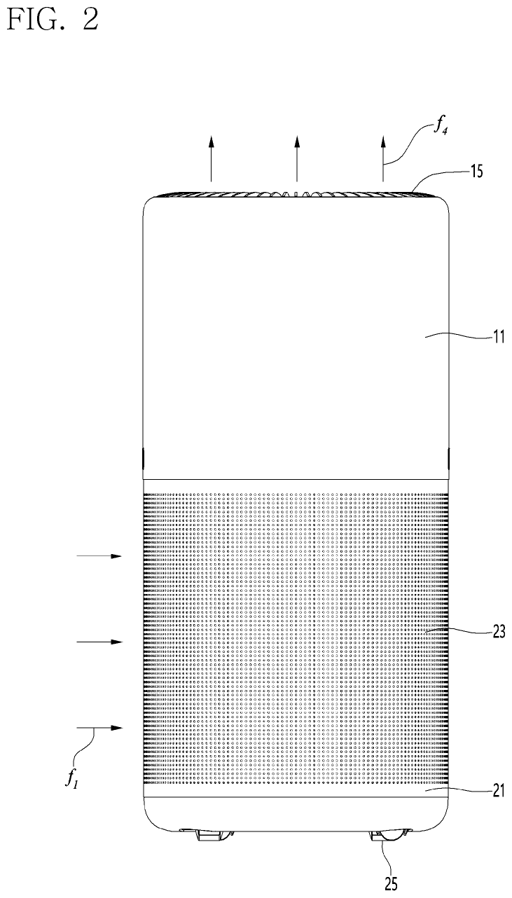

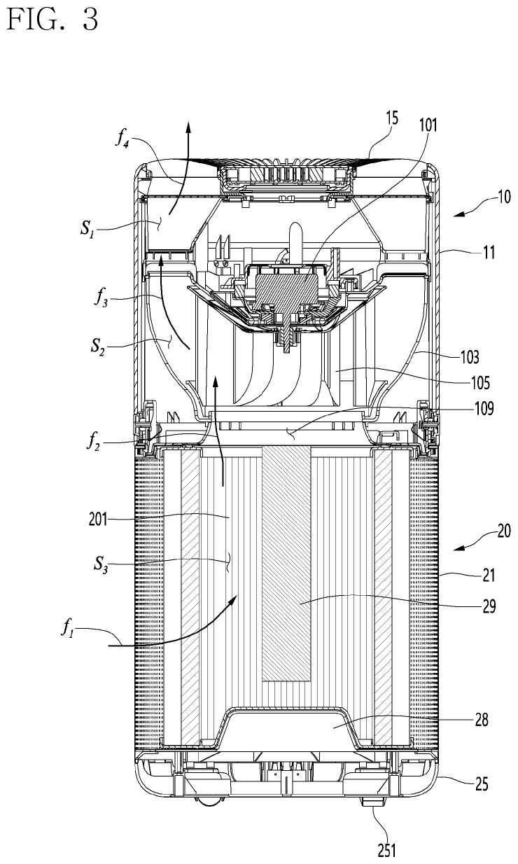

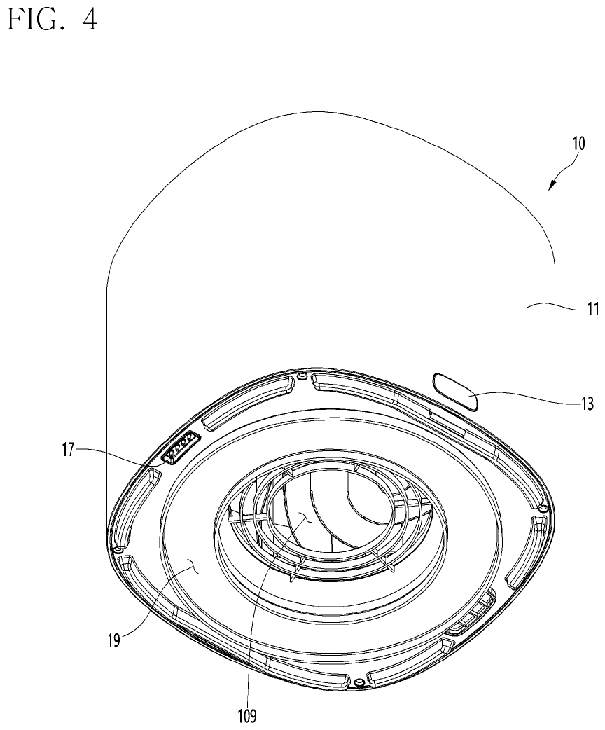

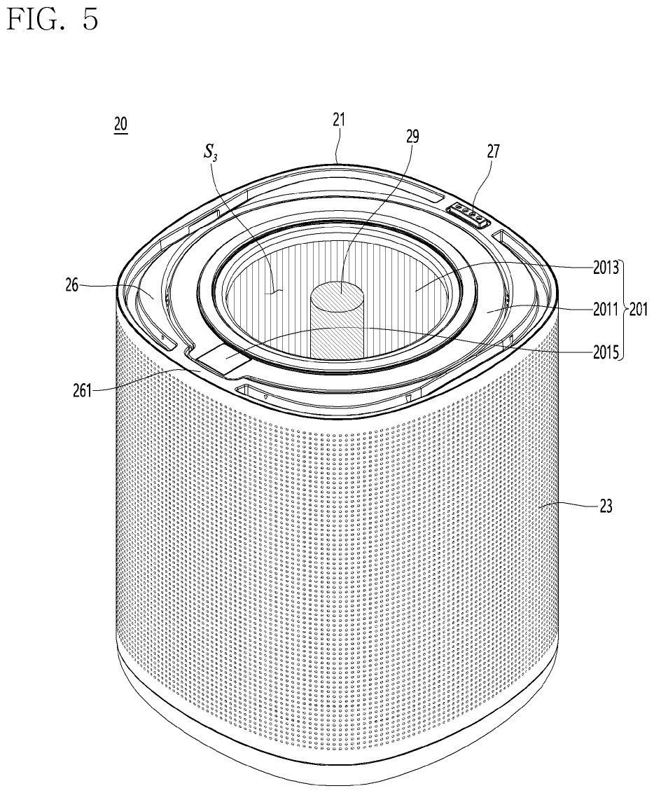

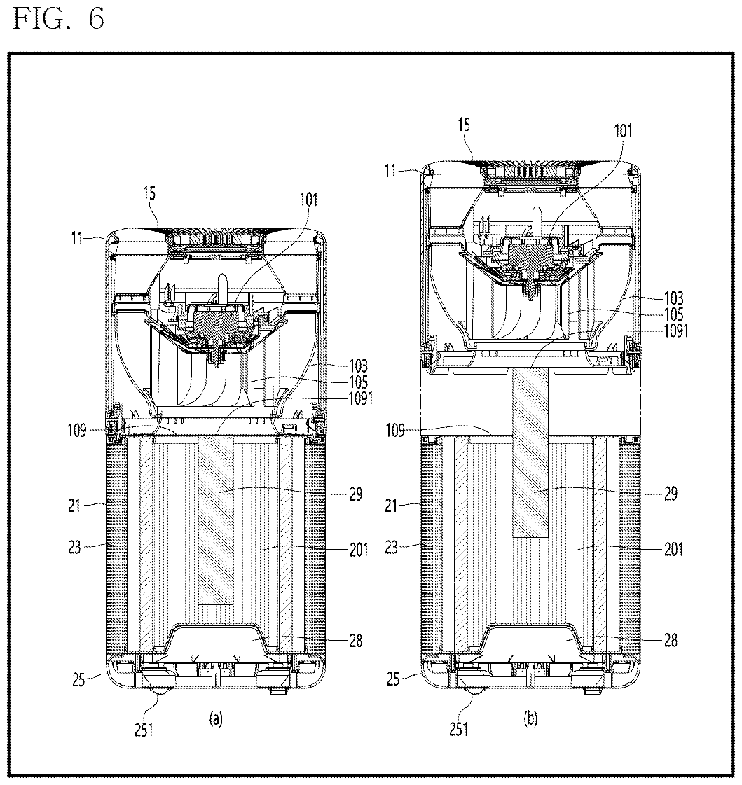

FIG. 1 is a perspective view illustrating an air cleaner according to an embodiment of the present invention, FIG. 2 is a diagram illustrating a side surface of FIG. 1, FIG. 3 is a diagram illustrating a side cross-sectional surface of FIG. 1, FIG. 4 is a diagram illustrating a first body of FIG. 1, FIG. 5 is a diagram illustrating a second body of FIG. 1, and FIG. 6 is a diagram illustrating an embodiment of coupling and detachment of the first body and the second body of FIG. 1.

Hereinafter, the present invention will be described with reference to FIGS. 1 to 6.

An air cleaner 1 according to an embodiment of the present invention may include bodies 10 and 20, a suction unit 23, a discharge unit 15, a fan module, a filter unit 201, and a light source unit 29.

The bodies 10 and 20 may form an appearance of the air cleaner 1. The body may include a first body 10 and a second body 20. The first body 10 has a discharge unit 15 formed on one surface and forms a space for accommodating the fan module therein.

The first body 10 and the second body 20 may be provided in various shapes. As an example, the cross-section may be provided in a rectangular shape or may be provided in a cylindrical shape.

The first body 10 and the second body 20 of the present embodiment may be provided in a shape close to a cylindrical shape with curved corners.

The suction unit 23 may be formed at various positions communicating with an inside of the second body 20, and the discharge unit 15 may be formed at various positions communicating with the inside of the first body 10.

The discharge unit 15 of the air cleaner 1 of the present embodiment is formed on an upper surface of the air cleaner 1 and the suction unit 23 is formed on a lower side of the air cleaner 1. That is, the discharge unit 15 is formed on the upper surface of the first body 10, and the suction unit 23 is formed on the second body 20.

As in the structure described above, it is preferable that a discharge port be formed on an upper side of the air cleaner in order to discharge the purified air to a user's real life space and at the same time expand a diffusion area of the discharged air. Therefore, the discharge port of the present embodiment is provided on an upper surface of the air cleaner.

The air cleaner 1 of the present embodiment is an air cleaner having an upper discharge structure that sucks f1 outside air from the lower portion, purifies the outside air in the air cleaner 1, and then discharges f4 the purified air from an upper portion of the air cleaner 1.

The first body 10 may be formed by a first housing 11 that forms an accommodation space therein and has a shape close to a cylindrical shape with curved corners. That is, the first housing 11 may form the overall shape of the first body 10.

The first body 10 is located above the air cleaner 1. The first body 10 is located relatively higher than the second body 20, and a lower end of the first body 10 may be in contact with an upper end of the second body 20, and the first body 10 and the second body 20 may be coupled. Of course, the first body 10 and the second body 20 may be separably coupled.

The discharge unit 15 may be formed on the upper surface of the first body 10. In more detail, the discharge unit 15 of the present embodiment may form a part of the upper surface of the first body 10, and the other part of the upper surface of the first body 10 may be provided with a display 14. Of course, the discharge unit 15 may be formed on the entire upper surface of the first body 10.

The display 14 may display air quality of an indoor space in which the air cleaner 1 is currently provided. Of course, the display 14 may represent various types of information such as the current time, the temperature and humidity of the space, the air cleaning mode input to the air cleaner, and the operating time of the air cleaner. The user may be able to control the above-described elements through the display 14.

As described above, when the display 14 is provided on a part of the upper surface of the first body 10, the display 14 may be formed in the center of the upper surface of the first body 10, and the discharge unit 15 may be formed on the remaining area of the upper surface of the first body 10 along the circumference of the display 14. This is because the amount of air that is purified through the air cleaner 1 and discharged may be increased when the area of the discharge unit 15 is widened as much as possible.

Therefore, as described above, the entire upper surface of the air cleaner may be provided as the discharge unit, and the above-described display may be formed on some of the side surfaces of the air cleaner.

A button 13 may be provided on one side of the first housing 11. The button 13 may perform coupling and detachment of the first body 10 and the second body 20. For example, the user may press the button 13 to separate the first body 10 from the second body 20 while releasing the coupling between the first body 10 and the second body 20.

The suction unit 23 of the present embodiment may be formed by the second housing 21 having a shape close to a cylindrical shape in which the second body 20 forms an accommodation space therein and corners are curved. That is, the second housing 21 may form the overall shape of the second body 20.

The suction unit 23 is a part into which outside air is introduced. The suction unit 23 is preferably formed in a large area in order to increase the amount of air sucked. Accordingly, the suction unit 23 may be formed along the circumference of the second body 20 in order to increase the amount of air sucked. The suction unit 23 is formed with a plurality of through holes, and the through holes may be formed along the circumference of the second housing 21.

The suction unit 23 of the present embodiment may be provided at a 360° whole surface along the outer circumferential surface of the second body 20 to suck polluted outside air in all directions. Accordingly, the air purifier may suck the polluted outside air from all directions without being restricted due to the installation environment.

A base 25 may be separably coupled to a lower side of the second body 20. The base 25 may support the air cleaner 1 from the ground. A support member 251 may be provided at a lower end of the base 25. As an example, the support member 251 may be provided in the form of a wheel to support the air cleaner 1 from the ground and facilitate the movement of the air cleaner 1.

Hereinafter, the internal configuration of the air cleaner 1 and the flow of air passing through the air cleaner 1 according to an embodiment of the present invention will be described with reference to FIGS. 3 to 5.

The filter unit 201 and a light source unit 29 may be provided inside the second body 20. The filter unit 201 may be provided at a position facing the suction unit 23 while being spaced apart from the suction unit 23 by a predetermined distance inside the second body 20.

The filter unit 201 may be positioned such that the filter 2021 is provided along the inner circumference of the second body 20 as the second body 20 is provided in a cylindrical shape. Accordingly, a filter 2021 of the filter unit 201 may be provided in a cylindrical shape.

The filter unit 201 may include a filter 2013 that is provided in a cylindrical shape, forms at least one layer, and filters air introduced from the outside, a filter frame 2011 that provided at an upper end of the filter 2013 to fix the shape of the filter 2013, and a protrusion 2015 protruding toward the outside of the filter frame 2011.

The filter 2013 may be formed of various materials for effectively filtering the introduced outside air. The filter 2013 may be formed by attaching a plurality of filter members layer by layer in a direction toward the inside of the second housing 21. In this case, a photocatalyst may be applied to a surface of the filter member facing the light source unit 29.

The photocatalyst may be defined as a variety of materials that respond to light irradiated from the light source unit 29. More specifically, the photocatalyst is a material that promotes a chemical reaction by receiving light energy, and as the photocatalyst, titanium oxide (TiO₃), and the like may be employed.

The photocatalyst may cause a chemical reaction by light irradiated from the light source unit 29 to decompose harmful substances contained in air that has passed through the filter 2013.

The protrusion 2015 protrudes on the outside of the filter frame 2011, and thus, when the filter unit 201 is inserted into and coupled to the second housing 21, may fix the position of the filter unit 201. More specifically, a recession 261 engaged with the protrusion 2015 is formed on the upper surface of the second housing 21, and when the filter unit 201 is inserted into the second housing 21, the protrusion 2015 is fitted into the recession 261.

Meanwhile, a label-type separation member (not illustrated) may be provided around the upper circumference of the filter frame 2011 so that a user may lift and separate the filter unit 201 from the second body 20. One side of the separation member may be coupled to the circumference of the opened upper portion of the second body 20, and the other side of the separation member may have a shape protruding from the upper portion of the second body 20 toward the open direction.

Accordingly, a portion of the separation member may be exposed to the outside while the filter unit 201 is inserted into the lower body. In order to prevent the force from being biased to one side when the filter unit 201 is lifted, the separation member is preferably provided in a plurality of pairs around the open upper portion of the first body to face each other.

With this structure, when the filter inserted into the empty space of the second body 20 is to be separated from the second body 20 for replacement or cleaning reasons, it may be performed by lifting the externally exposed separation member.

A second contact surface 26 may be formed on the upper surface of the second housing 21. The second contact surface 26 may contact an end portion of the first housing 11 when the first body 10 is coupled to the second body 20.

The second contact surface 26 may be formed to extend inwardly from the upper circumference of the second body 20. The inner circumference of the second contact surface 26 is formed to correspond to the shape of the outer circumference of the filter unit 201. Accordingly, when the filter unit 201 is inserted into the second housing 21, an internal sealing rate of the second body 20 may be increased. As described above, as the internal sealing rate of the second body 20 is increased, the air cleaning area of the air cleaner may be enlarged.

The fan module is provided inside the first body 10 to form a flow of air flowing from the suction unit 23 to the discharge unit 15.

The fan module may include a driving unit 101 that forms a rotation shaft and generates driving force, a fan that receives power from the driving unit 101 and rotates about the rotation shaft to form the air flow, and a shroud 103 forming a space for accommodating the driving unit 101 and the fan and provided between the suction unit 23 and the discharge unit 15 to guide air sucked through the suction unit 23 to the discharge unit 15.

The fan may include a hub coupled to the rotation shaft of the driving unit and a plurality of blades 105 extending in a radial direction from the hub. In addition, the shroud may be provided with a fan suction port corresponding to the through part 109.

The hub may include a coupling hole into which the rotation shaft of the blower motor is inserted, and a guide part extending radially from the coupling hole to change a movement path of air sucked through the suction port. The guide part may be defined as a part of a lower end of the shroud 103.

That is, the outside air introduced from the suction unit 23 may move from the lower portion to the upper portion of the air cleaner 1 along the longitudinal direction of the air cleaner 1, spread radially by the fan module, and move the discharge unit 15. Accordingly, as an example of the present embodiment, as the fan, a four-flow fan may be employed.

Reviewing the flow of air inside the first body 10 in more detail, air may move from the lower portion to the upper portion of the air cleaner 1 in a direction parallel to the rotation shaft of the motor through the fan suction port, and a movement path may be changed in a lateral direction of the motor through the guide part. Therefore, the guide part may be provided in a conical shape through which the upper and lower portions penetrate, and the rotation shaft of the motor may be inserted into a portion through which the guide part penetrates, so the penetrating portion of the guide part may be defined as the coupling hole.

A first connection part 17 may be provided at a lower portion of the first housing 11. When the first body 10 is coupled to the second body 20, the first connection part 17 may be connected to the second connection part 27 provided on the upper portion of the second housing 21. As the first connection part 17 and the second connection part 27 are connected, the first body 10 may receive power from the outside through the second body 20.

Meanwhile, the through part 109 may be formed in the lower inner side of the first housing 11. The through part 109 may form a part of a flow path through which outside air introduced into the suction unit 23 of the second body 20 moves to the first body 10. That is, the first body 10 and the second body 20 may communicate with each other through the through part 109.

In addition, a first contact surface 19 may be formed on the lower surface of the first housing 11 along the circumference of the through part 109. The first contact surface 19 is a surface in contact with the upper surface of the second housing 21 or the filter frame 2011, and when the first body 10 is coupled or separated from the second body 20 by the first contact surface 19, the rigidity of the first body 10 may be maintained.

In addition, when the first body 10 and the second body 20 are combined, it is of course possible to increase the internal sealing rate of the air cleaner 1 by shielding unnecessary parts other than the flow path formed inside the air cleaner 1.

Meanwhile, the first body 10 and the second body 20 may be separably provided. In addition, the first body 10 may be provided to be compatible with the second body 20.

It is effective to set the amount of outside air sucked differently depending on the size of the space where the air cleaner is installed. Of course, it is possible to control the suction amount of air cleaner in a software manner, but as in this embodiment, when the second body 20 in which the filter is accommodated is provided to be replaceable and compatible with the first body 10, it is possible to adjust the physical suction amount of the air cleaner in a hardware manner.

Therefore, the air cleaner includes the first body 10 and the second body 20 according to the relative height difference, and the two bodies may be coupled to each other to be separated, and in particular, the second body 20 in which the light source unit is accommodated may be provided to be compatible with the first body 10.

The fan rotation speed of the air cleaner may be automatically set according to the air cleaning capacity of the second body 20. This is because the larger the air purification capacity of the second body 20, the wider the air purification range of the air cleaner, and therefore, it is preferable to increase the fan rotation speed.

As an example of the automatic setting of the fan rotation speed, there will be a method of controlling a fan module according to an air cleaning capacity of the body 20, and more specifically, a method of controlling a fan module through the connection of the first connection part 17 and the second connection part 27 according to an intrinsic value input to the first body 10.

As described above, the air cleaner 1 of this embodiment has a structure that sucks outside air from the lower circumference and discharges the outside air to the upper portion. More specifically, outside air is introduced (f1) into the inside of the second body 20 through the suction unit 23 by the airflow formed during the operation of the fan module.

The outside air introduced into the second body 20 is filtered by the filter unit 201, and the pollutant is sterilized by the light source unit 29 in an inner space S3 of the filter unit 201.

The air passing through the filter unit 201 and the light source unit 29 rises (f2) toward the first body 10 along the longitudinal direction of the second body 20. The external air is moved in a direction away from the axial direction of the motor by the four-flow fan motor, is guided along the inner surface of the shroud 103, and moves (f3) along the space S2 between the shroud 103 and the fan module.

Then, the outside air moves to the space S1 between the fan module and the discharge unit 15 and is discharged to the outside of the air cleaner 1 through the discharge unit 15.

The base 25 is coupled to the lower portion of the second body 20. Therefore, the air cleaner 1 may be formed by laminating the second body 20 and the first body 10 from the base 25 in this order.

The base 25 may include a protrusion 28.

The protrusion 28 is formed to protrude from the base 25 toward the first body 10. That is, the protrusion 28 protrudes from the base 25 to form a predetermined height.

When the base 25 and the second body 20 are coupled, the protrusion 28 is located in the inner space S3 of the filter unit 201. The protrusion 28 may be formed to correspond to the shape of the lower end of the filter unit 201. More specifically, as the filter unit 201 is provided in a cylindrical shape, the protrusion 28 may protrude from the base 25 while forming a circumference corresponding to the inner circumferential surface of the filter unit 201.

The axial movement of the filter unit 201 and the second body 20 may be fixed when the second body 20 is coupled to the base 25 due to the structure of the above-described protrusion 28.

The light source unit 29 may be provided inside the second body 20 to radiate light toward the inner side surface of the filter unit 201. The light source unit 29 may be coupled to the lower side of the first body 10 and may be formed to extend a predetermined length toward the base 25. The light source unit 29 may extend from the lower portion of the first body 10 to the position close to the top surface of the base 25.

Referring to FIG. 6, the light source unit 29 of the present embodiment may be provided in a cylindrical shape forming a predetermined length. As the light source unit 29 is provided in a cylindrical shape along the longitudinal direction of the second body 20, light may be evenly irradiated to the inner surface of the filter unit 201.

The light source unit 29 is preferably located in the center of the filter unit 201. As the second body 20, the filter unit 201, and the light source unit 29 of the present embodiment are provided in a cylindrical shape, the light source unit 29 may be provided at a position that shares a central axis with the second body 20 and the filter unit 201.

The light source unit 29 of the present embodiment may receive power when the first body 10, the second body 20, and the base 25 are combined.

The light generated from the light source unit 29 is irradiated 360° so as to reach the entire photocatalytic filter layer. The light source unit 29 may be formed of a lamp, an LED, or the like. In the case of a general photocatalyst, the light source unit 29 may be configured of an ultraviolet lamp or an ultraviolet LED, and in the case of a visible photocatalyst, the light source unit 29 may be configured of an ultraviolet lamp, an ultraviolet LED, a high-brightness lamp, or a high-brightness LED. In addition, the photocatalyst filter layer forming the inner side surface of the filter unit 201 may be formed by applying a photocatalyst on a support having a mesh structure such as urethane foam in order to minimize the portion acting as a resistance to the flow of the incoming air.

The air cleaner removes dust in circulation processes several times, and the air sterilization should be performed for an effective period of time. For this reason, when the air forms a fast airflow to secure the air cleaning ability, the sterilization ability is greatly reduced.

For the above reasons, in order to sterilize the air in the air cleaner, even if the air cleaning ability is reduced, a method that allows air to flow slowly or has very strong sterilization power needs to be used. According to the structure described above, the light source unit 29 is located in the center of the filter inner space S3 to directly irradiate the inner side surface of the filter unit 201 with light to improve the sterilization power, thereby preventing the sterilization ability from deteriorating while maintaining the output of the fan motor.

In addition, the light source unit 29 is coupled to the lower portion of the first body 10 and is lifted from the second body 20 together with the first body 10 when the first body 10 is separated from the second body 20, so the first body 10 may be separated from the second body 20 without the user directly contacting the light source unit 29, thereby minimizing the risk of a safety accident and facilitating the maintenance.

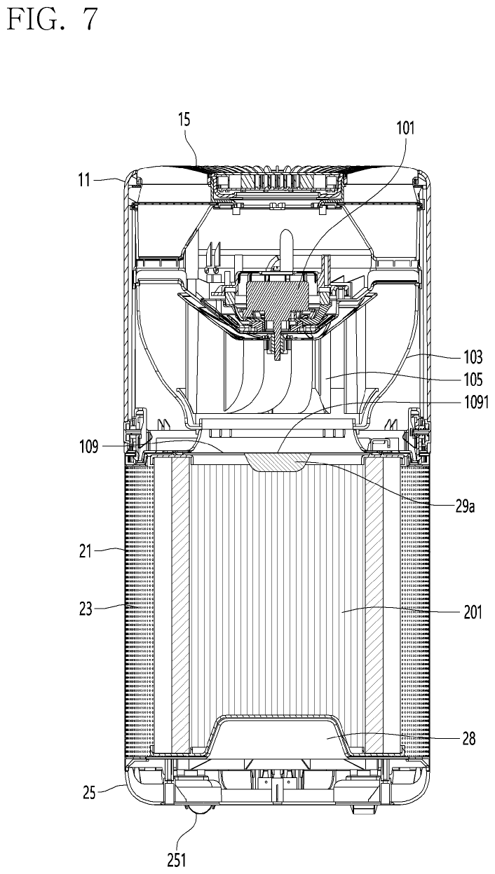

FIG. 7 is a side cross-sectional view of an air cleaner to which a light source unit according to another embodiment of the present invention is applied.

Referring to FIG. 7, a light source unit 29a of this embodiment is coupled to the lower side of the first body 10, and is formed to protrude from the lower side of the first body 10 toward the base 25. According to the structure of the present embodiment, the maintenance efficiency of the light source unit is increased.

This is because, when lifting the first body 10 by separating the first body 10 from the second body 20 for the maintenance of the light source unit, the first body 10 needs to be lifted from the second body 20 by the extended length of the light source unit.

However, according to the structure of the light source unit 29a of the present embodiment, it is difficult to evenly transmit light to the inner side surface of the filter unit compared to the structure of the light source unit 29 described above. Therefore, the light source unit 29a of this embodiment is advantageously applied when the height of the filter unit is low because the air cleaning area of the second body 20 is relatively small.

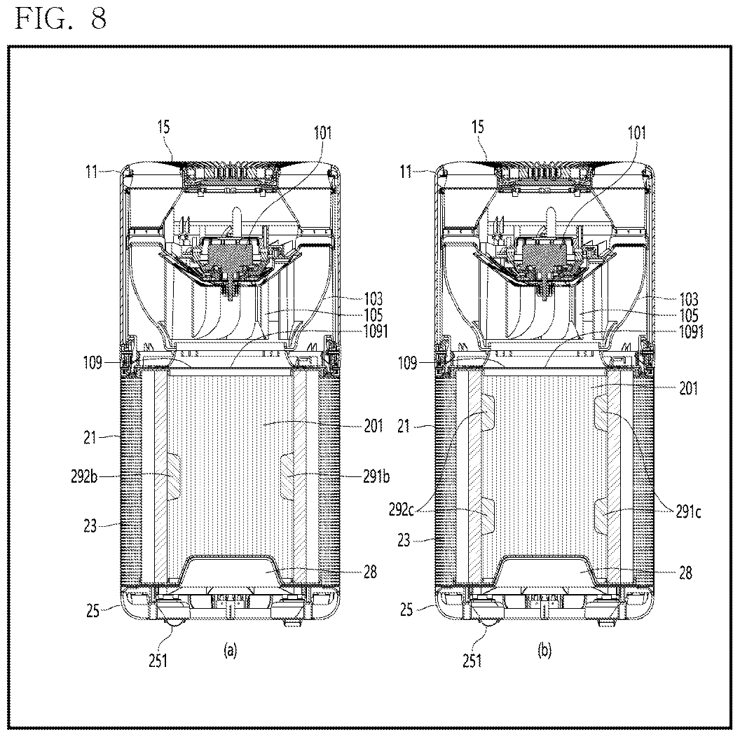

FIG. 8 is a side cross-sectional view of an air cleaner to which a light source unit according to another embodiment of the present invention is applied.

In FIGS. 8A and 8B, the light source unit is provided on the side of the filter unit and disposed at a position symmetrical with respect to the protrusion. Hereinafter, light source units 291b and 292b of FIG. 8A will be described as a reference.

The light source units 291b and 292b of the present embodiment may include a first light source unit 291b and a second light source unit 292b, and each light source unit may be provided at a position symmetrical to each other inside the second housing.

The first light source unit 291b of the present embodiment may irradiate light to a portion of the inner surface of the filter unit, and the second light source unit 292b may radiate light to the remaining portion of the inner surface of the filter unit.

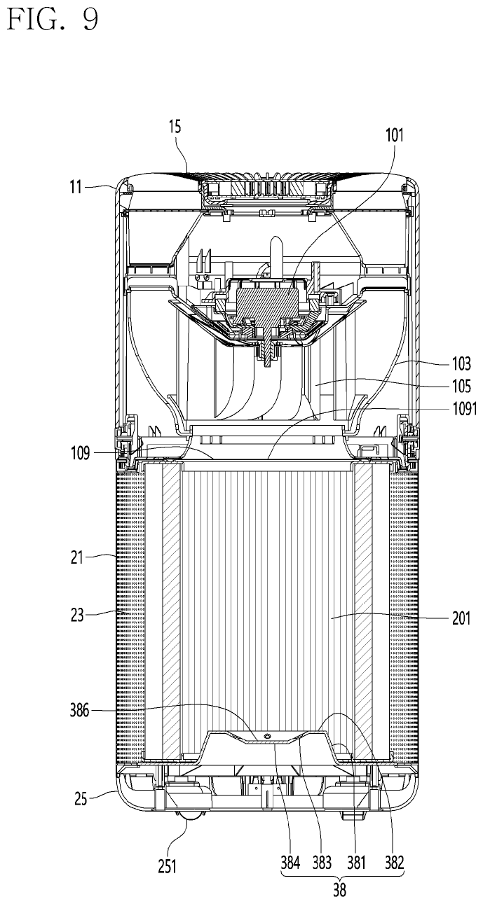

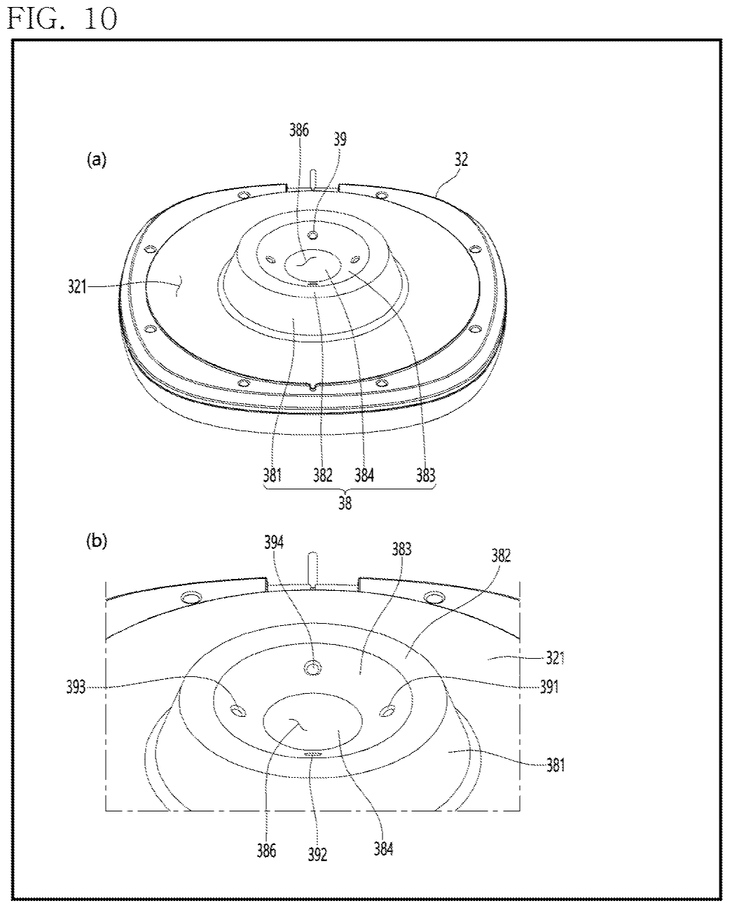

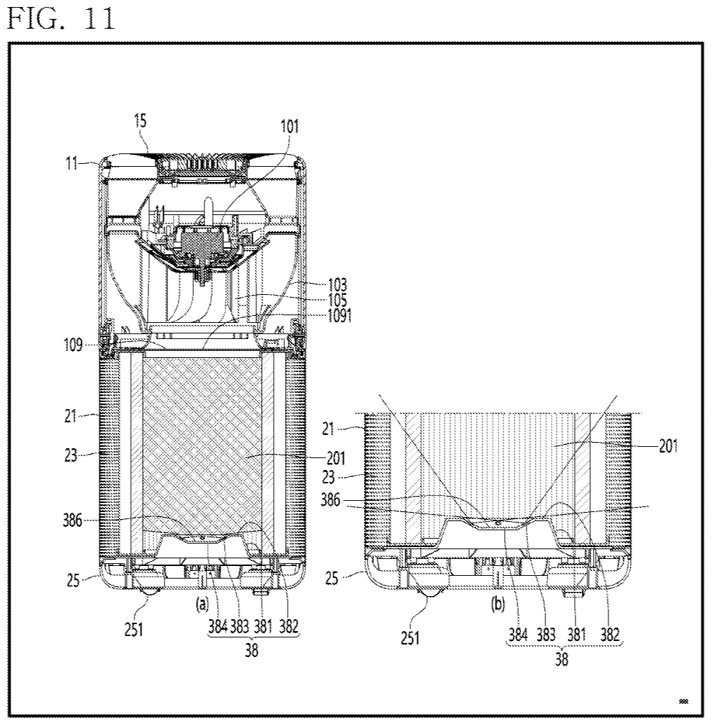

FIG. 9 is a side cross-sectional view of the air cleaner to which the light source unit is applied according to another embodiment of the present invention, FIG. 10 is a view illustrating the base of FIG. 9, and FIG. 11 is a view illustrating the sterilization area of the filter by the light source unit of FIG. 9.

Hereinafter, the present invention will be described with reference to FIGS. 9 to 11.

The base 32 of this embodiment forms a protrusion 38 and a filter support surface 321. The filter support surface 321 may be in contact with a lower portion of the filter unit to support the filter. Accordingly, the filter support surface 321 may be provided to correspond to the shape of the lower portion of the filter unit.

The light source unit 39 of this embodiment is provided on the base 32. More specifically, the base 32 includes a protrusion 38, and the protrusion 38 is formed to protrude from the base 32 toward the first body 10. That is, the protrusion 38 protrudes from the base 32 to form a predetermined height.

When the base 32 and the second body 20 are coupled, the protrusion 38 is located in the inner space S3 of the filter unit 201. The protrusion 38 may be formed to correspond to the shape of the lower end of the filter unit 201. More specifically, as the filter unit 201 is provided in a cylindrical shape, the protrusion 38 may protrude from the base 32 while forming a circumference corresponding to the inner circumferential surface of the filter unit 201.

The axial movement of the filter unit 201 and the second body 20 may be fixed when the second body 20 is coupled to the base 32 due to the structure of the above-described protrusion 38.

A recession 386 may be formed on the upper surface of the protrusion 38. The recession 386 may be formed to be recessed toward the base 32 from the upper surface of the protrusion 38.

The light source unit 39 may be provided on a side surface forming the recession 386. A side surface of the recession 386 may be inclined. The inclination may be formed in a direction from the upper surface of the protrusion 38 toward the center of the protrusion 38.

More specifically, the recession 386 may include a first surface 381, a second surface 382, a third surface 383, and a fourth surface 384. The first surface 381 may be defined as a surface extending upwardly from the base 32. That is, the height at which the first surface 381 is formed may correspond to the height at which the recession 386 protrudes from the base 32.

The second surface 382 may be defined as a surface extending from the first surface 381 in a direction in which the cross-sectional area of the protrusion 38 decreases. That is, the second surface 382 may correspond to the front end surface of the recession 386.

The third surface 383 may be defined as a surface inclined toward the base 32 from the second surface 382. In the present embodiment, the inclination formed by the third surface 383 refers to a direction from the second surface 382 toward the center of the recession 386.

The fourth surface 384 may refer to a recessed surface of the recession 386. Accordingly, the third surface 383 is formed along the circumference of the fourth surface 384.

The light source unit 39 is preferably provided on the third surface 383. This is because, as the third surface 383 is formed to be inclined, when the light source unit 39 is provided on the third surface 383, light may be directly irradiated toward the inner surface of the filter unit.

In addition, as described above, in order to directly irradiate light toward the inner surface of the filter unit, the light source unit 39 needs to be disposed on an inclined surface, and when the light source unit 39 is provided on the third surface 383 as in the present embodiment, structural factors that block light can be minimized.

Meanwhile, a plurality of light source units 39 may be provided on the third surface 383 spaced apart from by a predetermined interval.

The light source unit 39 of the present embodiment may include a first light source member 391, a second light source member 392, a third light source member 393, and a fourth light source member 394. Each of the light source members may be disposed at a position facing each other at a 90°.

By the arrangement of the light source member described above, the first light source member 391 covers an area of 25% of the inner surface of the cylindrical filter, the second light source member 392 covers an area of 25% of the inner surface of the cylindrical filter, the third light source member 393 covers an area of 25% of the inner surface of the cylindrical filter, and the fourth light source member 394 covers an area of 25% of the inner surface of the cylindrical filter, thereby increasing the sterilization effect by the light source by irradiating light directly to the inner surface of the filter unit while evenly irradiating light.

In addition, an overlapping area may occur among the areas irradiated by the respective light source members, and it is obvious that the sterilization effect by the light source unit increases as the overlapping area becomes wider.

On the other hand, as the light source unit 39 is provided on the base 32, the efficiency of the light source unit 39 is increased by directly receiving external power from an electric wire connected to the base 32.

In addition, after separating the second body from the base 32 for management of the light source unit 39, the light source unit 39 may be operated while the base 32 is supported on the ground, so the management of the light source unit 39 is easy.

On the other hand, since the removable IoT module is attached to the air cleaner of the present embodiment, the IoT environment may be built through the module. The IoT module is a module that allows a user to control an existing product existing in the home as desired.

The IoT system may be installed in a space where a user lives, and a plurality of home appliances capable of changing the state of outside air of the space may be installed in the space. Of course, the home appliance may include various home appliances that may be manipulated in the space by a user as well as changing the state of the outside air. As an example of the home appliance, the air cleaner of this embodiment may be applied.

The wireless communication method between the home appliance and the terminal is very diverse. For example, Bluetooth, ZigBee, Wifi, etc. are the most used, and various standards such as power line communication and wireless power communication may be used.

Although various embodiments of the present invention have been disclosed for illustrative purposes, those skilled in the art will appreciate that various modifications, additions and substitutions are possible, without departing from the scope and spirit of the invention as disclosed in the accompanying claims. Accordingly, the scope of the present invention is not construed as being limited to the described embodiments but is defined by the appended claims as well as equivalents thereto.

1. An air cleaner, comprising:
a body forming an appearance;
a suction unit formed on the body and introduced with outside air;
a discharge unit formed on the body and discharging the air introduced through the suction unit;
a fan module provided inside the body and forming a flow of air introduced into the suction unit and discharged to the discharge unit;
a filter unit provided inside the body to filter the air introduced into the suction unit; and
a light source unit provided inside the body and irradiating light toward an inner side surface of the filter unit.

2. The air cleaner of claim 1, wherein the body includes:
a first body having a discharge unit formed on one surface and forming a space for accommodating the fan module; and
a second body having a suction unit formed on one surface and forming a space for accommodating the filter unit and the light source unit.

3. The air cleaner of claim 2, wherein the first body is separably coupled to an upper portion of the second body.

4. The air cleaner of claim 3, wherein the suction unit is formed along an outer circumference of the second body, and the filter unit is spaced apart from the suction unit by a predetermined distance inside the second body and is provided at a position facing the suction unit.

5. The air cleaner of claim 2, further comprising:
a base separably coupled to a lower portion of the second body to support the air cleaner.

6. The air cleaner of claim 5, wherein the light source unit is coupled to the first body to extend by a predetermined length toward the base.

7. The air cleaner of claim 5, wherein the light source units are coupled to an inner side of the second body and are each provided at positions facing each other with respect to a central axis of the base.

8. The air cleaner of claim 5, wherein the light source unit is provided on the base.

9. The air cleaner of claim 8, wherein the base includes a protrusion formed to protrude toward the first body, and the light source unit is provided in the protrusion.

10. The air cleaner of claim 9, wherein the protrusion includes a recession that is recessed from an upper surface of the protrusion toward the base, and
the light source unit is provided on a side surface forming the recession.

11. The air cleaner of claim 2, wherein the second body is provided in a cylindrical shape, the filter unit is provided in a cylindrical shape corresponding to a curvature of the second body, and the light source unit is located in a hollow formed by the second body.

12. The air cleaner of claim 11, wherein the light source unit is coupled to the first body and is provided in a cylindrical shape extending by a predetermined length.

13. The air cleaner of claim 12, wherein central axes of the second body, the filter unit, and the light source unit are the same.
